(19) United States
(12) Patent Application Publication
(10) Pub. No.: US 2021/0113727 A1
(43) Pub. Date: Apr. 22, 2021

(54) SYSTEM FOR DETECTING AND COMBATING URINARY CATHETER-DWELLING BACTERIA

(71) Applicant: Analog Devices International Unlimited Company, Limerick (IE)

(72) Inventors: J. Brian HARRINGTON, Revere, MA (US); Pravinkumar ANGOLKAR, Bangalore (IN); Hari CHAUHAN, Lexington, MA (US); Craig BREEN, Arlington, MA (US); Alain Valentin GUERY, Boston, MA (US)

(73) Assignee: Analog Devices International Unlimited Company, Limerick (IE)

(21) Appl. No.: 17/070,642

(22) Filed: Oct. 14, 2020

Related U.S. Application Data
(60) Provisional application No. 62/942,634, filed on Dec. 2, 2019.

(30) Foreign Application Priority Data
Oct. 17, 2019 (IN) 201941042040

(51) Int. Cl. A61L 2/24; A61M 25/00; A61L 2/10

(52) U.S. Cl. CPC A61L 2/24; A61M 25/0017; A61L 2/10; A61M 2205/3334; A61M 2205/3368; A61L 2202/24; A61M 2205/70; A61M 2205/3584; A61M 2205/6054; A61L 2202/14; A61L 2202/11; A61M 2025/0019

(57) ABSTRACT

One embodiment is a urinary catheter system comprising an optical flow tube for connecting a catheter tube to a collection tube; and a disinfection and detection device configured to enclose the optical flow tube for detecting and destroying bacteria within the optical flow tube.

### CROSS REFERENCE TO RELATED APPLICATIONS

This application claims the benefit of and priority from an International Application No. IN 201941042040 filed 17 Oct. 2019 entitled, “SYSTEM FOR DETECTING AND COMBATING URINARY CATHETER-DWELLING INFECTIONS”, and from U.S. Provisional Application No. 62/942,634 filed 2 Dec. 2019 entitled, “SYSTEM FOR DETECTING AND COMBATING URINARY CATHETER-DWELLING INFECTIONS”, the disclosures of both of which are hereby incorporated by reference herein in its entirety.

### FIELD OF THE DISCLOSURE

This disclosure relates generally to the field of urinary catheters and, more particularly, to a system for detecting and combating urinary catheter-dwelling bacteria.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts, in which:

FIGS. 1A-1B illustrate various forms and features of urinary catheters for use in catheterizing a patient in accordance with embodiments described herein;

FIG. 2 illustrates an example urinary catheter system for detecting and combating urinary catheter-dwelling bacteria in accordance with embodiments described herein;

FIG. 3 is a perspective view of an example optical flow tube of a urinary catheter system for detecting and combating urinary catheter-dwelling bacteria in accordance with embodiments described herein;

FIGS. 4 and 5 are schematic diagrams of an example optical flow tube of a urinary catheter system for detecting and combating urinary catheter-dwelling bacteria in accordance with embodiments described herein;

FIG. 6 illustrates interaction between a flow tube and a clamshell spectrometer comprising a urinary catheter system for detecting and combating urinary catheter-dwelling bacteria in accordance with embodiments described herein;

FIG. 7 is a schematic diagram of a spectrometer of a urinary catheter system for detecting and combating urinary catheter-dwelling bacteria in accordance with embodiments described herein;

FIG. 8 illustrates interaction between a spectrometer of a urinary catheter system for detecting and combating urinary catheter-dwelling bacteria and cloud services in accordance with embodiments described herein;

FIGS. 9A and 9B illustrate graphs of example UV dosages for destroying bacteria in a urinary catheter system for detecting and combating urinary catheter-dwelling bacteria and cloud services in accordance with embodiments described herein;

FIG. 10 is another illustration of an optical flow tube of a urinary catheter system for detecting and combating urinary catheter-dwelling bacteria and cloud services in accordance with embodiments described herein; and

FIG. 11 illustrates a flow tube and a snap on spectrometer of a urinary catheter system for detecting and combating urinary catheter-dwelling bacteria and cloud services in accordance with embodiments described herein.

### DESCRIPTION OF EXAMPLE EMBODIMENTS OF THE DISCLOSURE

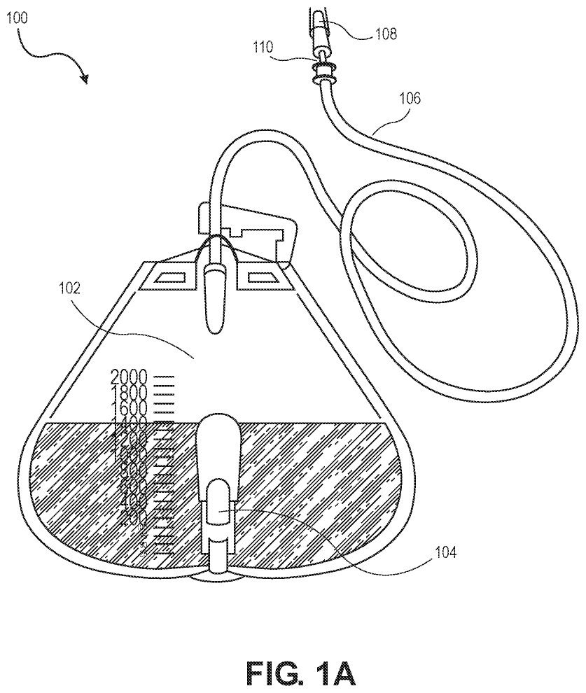

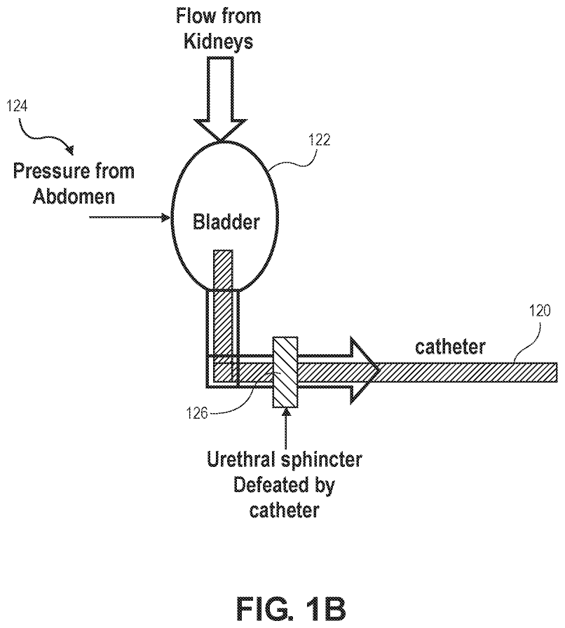

FIG. 1A illustrates a conventional catheter system 100 with which a patient may be catheterized. As shown in FIG. 1A, catheter system 100 includes a urine drainage bag 102 for collecting urine, a spout 104 for emptying urine from the drainage bag 102, a catheter tube 106 for transporting urine to the urine drainage bag 102, a connection adapter 108, and a connector 110 between the catheter (connection adapter 108) and catheter tube 106. It will be recognized that catheterized patients are highly susceptible to developing urinary tract infections (“UTIs”) as a result of bacteria that colonize in a catheter system, such as the system 100. UTIs are harder to diagnose in catheterized patients because the patients do not exhibit typical symptoms. When a UTI is suspected in a patient, laboratory tests may be performed.

Bacteria diffuse within a catheter through urine or travel through biofilm that forms inside the catheter, leading to decreased antibiotic efficacy. Biofilms that form inside the catheter block the light, thus impeding detection of bacteria as well as protecting the bacteria from ultraviolet C irradiation (“UVC”) that might otherwise be effective in ridding the catheter of bacteria.

A urinary catheter may be “urethral,” in which case it is inserted through the urethra into the bladder of the patient, or “suprapubic,” in which case it is inserted through the stomach directly into the bladder of the patient. Often, catheters are inserted to prevent blockages of urine flow. The catheter extends into the bladder and urine flow is expected to be lower flow and have a larger duty cycle than under normal (non-catheterized) circumstances. It will be recognized that urine flow back into the bladder should be avoided. Bacteria likely migrate by diffusion through the catheter. The catheter system should be closed to prevent infection. FIG. 1B is a representative illustration of a catheter 120 inserted into a bladder 122 of a patient 124 through the patient's urethra 125. In the embodiment illustrated in FIG. 1B, a urethral sphincter 126 of the patient 124 may be defeated by the catheter 120, allowing free flow of urine from the bladder 122 through the catheter 120.

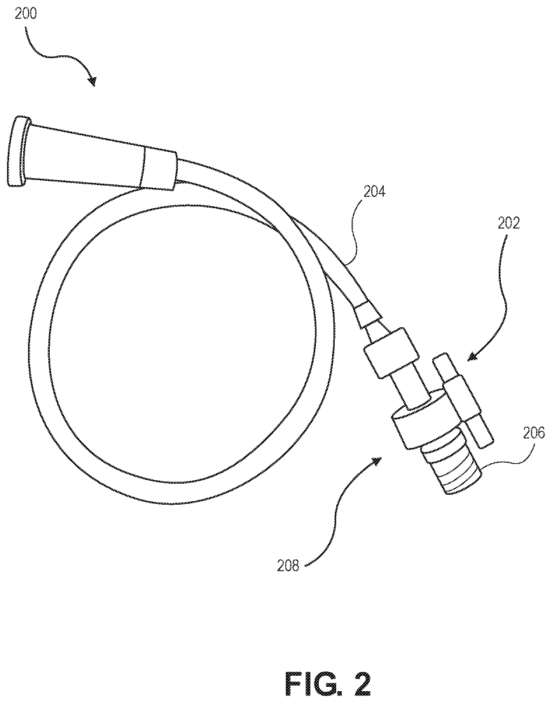

A goal of embodiments described herein is to provide a system that continuously detects and combats bacteria growth in a urinary catheter system. In one embodiment, the system includes two primary subsystems: (1) an optical flow tube; and (2) a disinfection/detection instrument (e.g., a spectrometer). In accordance with embodiments described herein, as illustrated in FIG. 2, a urinary catheter system 200 includes an optical flow tube 202, which replaces the catheter-collection tube connector 110 shown in FIG. 1A. More particularly, and as will be described in greater detail hereinbelow, the optical flow tube 202 connects a catheter tube 204 to a collection tube 206 and provides an optimized spectral environment and an acoustic coupler. The optical flow tube 202 prevents air bubble formation and provides flow and temperature measurement via sensors. In certain embodiments, the optical flow tube 202 is disposable. As will also be described in greater detail hereinbelow, a disinfection/detection instrument 208 encloses the optical flow tube 202 and disrupts biofilm formation and irradiates and monitors bacteria. More particularly, the disinfection/detection instrument 208 inhibits biofilm formation, kills bacteria, performs spectral urinalysis, and collects urine flow and temperature data. Additionally, the disinfection/detection instrument 208 may transmit data to cloud services and provide a basic user interface. Unlike the optical flow tube 202, the disinfection/detection instrument 208 is designed to be reusable.

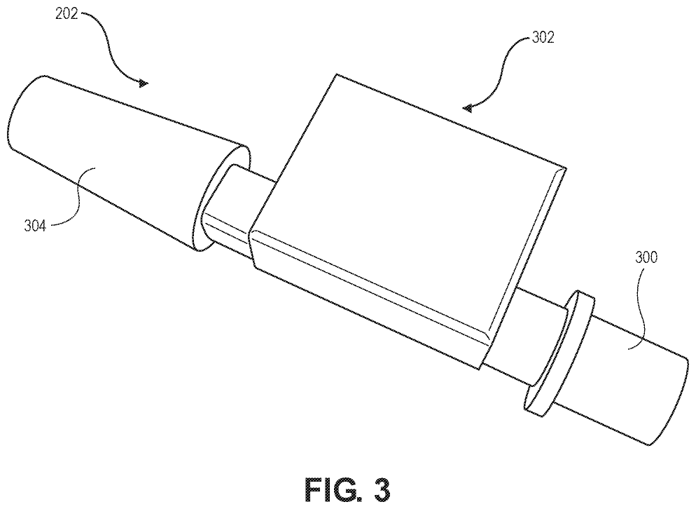

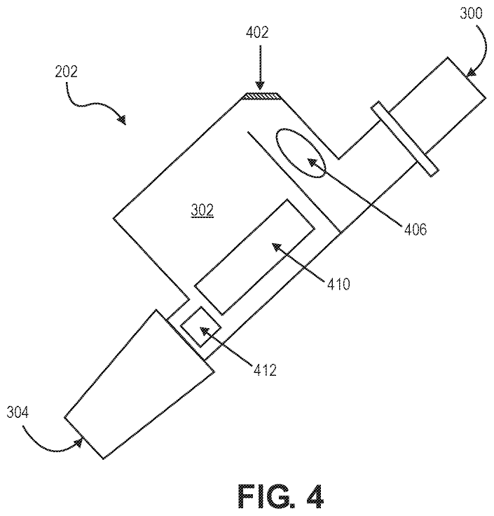

FIG. 3 is a perspective view of an embodiment the optical flow tube 202, including a collection tube connector 300, an optical cell 302, and a catheter connector 306. FIG. 4 illustrates a more detailed schematic view of an embodiment of the optical flow tube 202. As shown in FIG. 4, in addition to the collection tube connector 300, optical cell 302, and catheter connector 306, the optical flow tube 202 includes a permeable membrane 402, a bubble trap 404, an acoustic coupler 406, a near infrared (“NIR”) antireflective coating 410, and a UVC antireflective coating 412. In accordance with features of embodiments described herein, the optical flow tube is optically transparent at critical wavelengths of 255-275 nm (disinfection), 740-1100 nm (detection) and 1500-2500 nm (detection). UVC light is known to inhibit and/or destroy E. coli and other bacteria, which can be detected in the visible spectrum (VIS)-NIR range. The permeable membrane 402 comprises a bubble trap 404 for preventing air bubble formation. The acoustic coupler 406 captures and propagates acoustic energy to inhibit biofilm formation. In certain embodiments, the optical flow tube 202 is constructed of low-cost materials (e.g., PVC or PET) and is disposable.

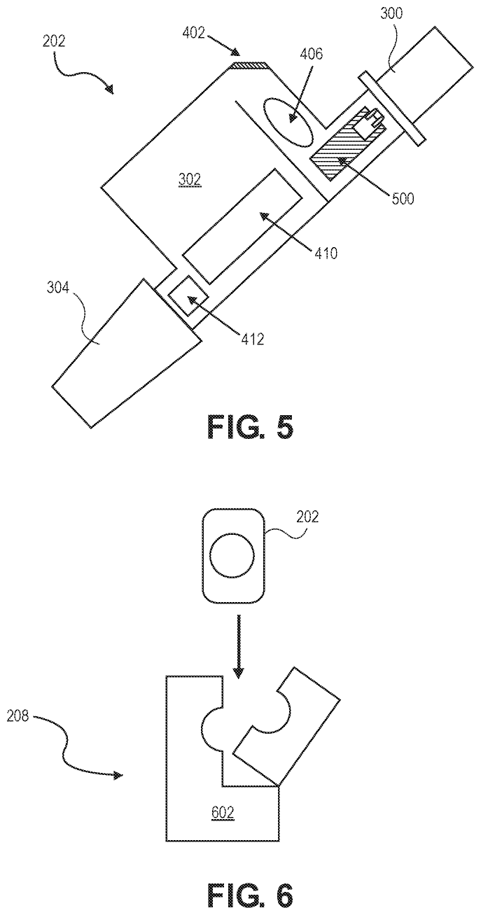

FIG. 5 illustrates a more detailed schematic view of the optical flow tube 202. In accordance with alternative embodiments described herein, as shown in FIG. 5, the optical flow tube 202 may optionally include optional urine flow and temperature measurement system 500. The flow and temperature measurement system 500 includes sensors for gauging the flow rate and temperature of urine flow through the optical flow tube 202, which may be useful in cases in which the flow rate affects detection scans and also enables the optical flow tube 202 to be used in monitoring kidney health. The flow and temperature measurement system 202 may be queried and powered by a radio frequency identification (RFID) reader included with a disinfection/detection system, as described below.

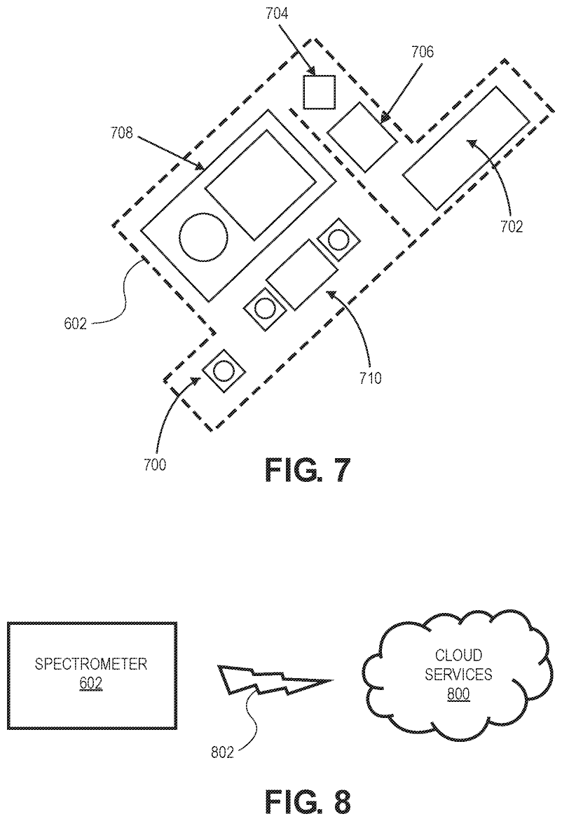

As shown in FIG. 6, in certain embodiments, the optical flow tube 202 may be inserted into and/or enclosed within disinfection/detection system 208 comprising a clamshell spectrometer 602 having a reflective interior. The spectrometer 602 performs spectral measurement over the VIS-NIR band and provides an optimized optical environment for the optical flow tube 202 by blocking ambient light and integrating excitation light sources. As shown in FIG. 7, the spectrometer 602 includes an ultraviolet-C (UVC) light emitting diode (LED) 700 for disinfecting the optical flow tube 202, thereby protecting the patient from infection and increasing the apparent detection limit. The spectrometer 602 further includes an ultrasound transmitter for inhibiting biofilm growth and orientation measurement for bubble prevention and detection. Referring also to FIG. 8, the spectrometer 602 also provides a gateway to cloud services 800 via a wireless connection 802 and an RFID reader 702 for collecting flow and temperature measurement data from and powering the sensors of the flow and temperature measurement system 500 (FIG. 5). The clamshell device blocks ambient light and provides VIS-NIR light integration, provides an accelerometer for orientation measurement (including bubble avoidance and detection), deters biofilm growth via ultrasound, and provides a UVC light source to disinfect the catheter.

As shown in FIG. 7, in addition to the RFID flow and temperature reader 702 and the UVC LED 700, the spectrometer 602 further includes an accelerometer 704, a piezoelectric or microelectromechanical system (“MEMS”) speaker 706, a VIS LED and photodetector (PD) 708, and IR LEDs and PD 710. The spectrometer 602 may be a dual spectrometer system providing energy in wavelength ranges of 740-1100 nm and 1500-2500 nm for bacterial detection. In certain embodiments, UVC LED 700 covers 275 nm and may be implemented using an Inolux IN-C33DTDU1 for bacterial disinfection. In certain embodiments, the piezoelectric or MEMS speakers may operate 706 at 70 KHz and the accelerometer 704 may be a 1 axis accelerometer for providing orientation information. The RFID flow and temperature reader 702 is optional and may include an antenna and may power and read the flow and measurement sensors of the optical flow tube 202.

FIG. 8 illustrates interaction between the spectrometer 602 and cloud services 800 via wireless connection 802. In certain embodiments, cloud services 800 perform spectrometer calibration, store spectral scan and sensor data, run chemometric models on the data to perform urine analysis, perform sensor fusion algorithms to amend and complement insights from NIR, accelerometer, and flow and temperature measurement sensors, and provide remote access to all collected data in compliance with HIPPA regulations.

Although embodiments have been described herein with reference to urine, the embodiments may be applied to any liquids. Additionally, a gravity system may be included to manage urine (or other liquid) flow. The optical flow tube 202 may be implemented using a simple flow tube or no flow tube at all, in which case the spectrometer 602 may be implemented as a clip-on case. A camera may be provided in some embodiments and analytics may be cloud-based or local. Moreover, a chemical sensor array may be provided and a fiber optic surface plasmon resonance (“SPR”) system may be provided at the catheter tip.

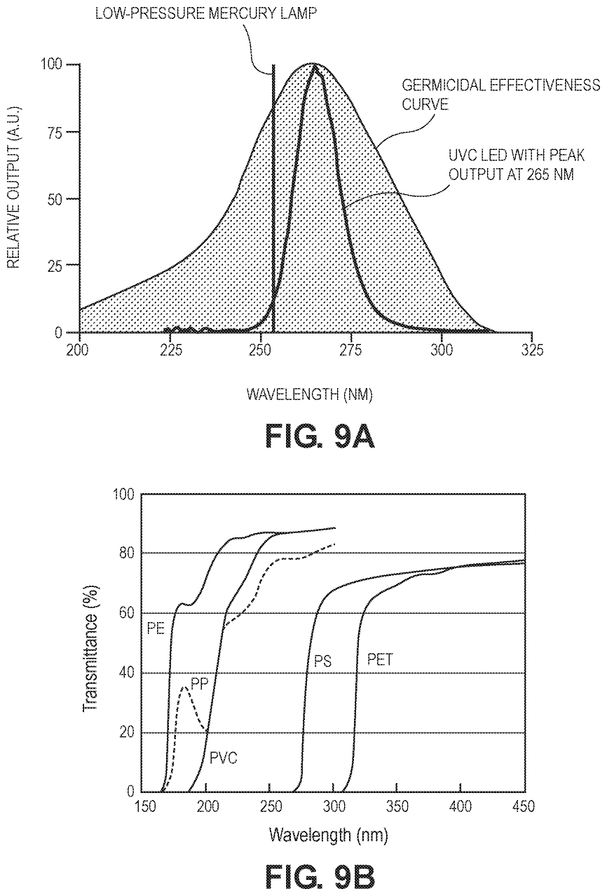

FIGS. 9A and 9B illustrate graphs of example UV dosages for destroying bacteria in the catheter system.

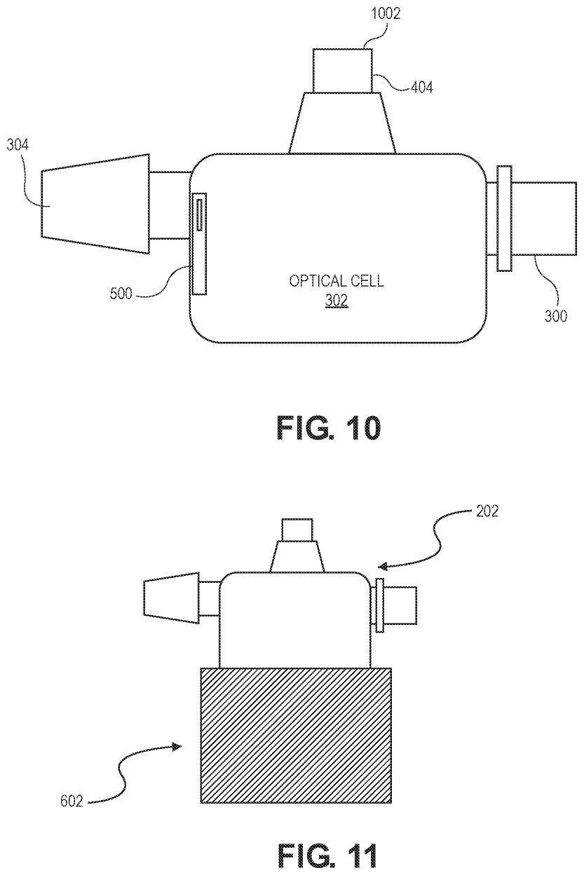

FIG. 10 illustrates another view of the optical flow tube 202 of catheter system 200 described herein. As previously described, in the system 200, catheter connecter 304 connects optical cell 302 to catheter tubing 1000. The optical cell 302 includes flow and temperature measurement sensors 500, a syringe luer lock 1002, bubble trap 404, collection tube connector 300 for connecting optical cell 302 to connection tubing 1004. The optical flow tube 202 is optically transparent in NIR range, rejects bubble formation, and is low cost and therefore disposable. The flow tube 202 may also measure flow rate and temperature to gauge urine flow and may be queried and powered by an RFID reader included within the spectrometer.

FIG. 11 illustrates another view of the spectrometer 602 as connected to the optical flow tube 202. As previously noted, the spectrometer 602 includes an interior reflective coating with an optical window. The spectrometer 602 performs spectral measurement over the NIR band, blocks ambient light, and provides NIR reflectivity for light collection. The spectrometer 602 also provides a gateway to cloud services and an RFID reader thereof collects flow and temperature data and powers flow and temperature sensors of the optical flow tube. The spectrometer 602 includes an accelerometer for measuring orientation and may be battery powered in some embodiments.

EXAMPLE 1 is a fluid catheter system including an optical flow tube for connecting a catheter tube to a collection tube; and a disinfection and detection device configured to enclose the optical flow tube for detecting and destroying bacteria within the optical flow tube.

In EXAMPLE 2, the fluid catheter system of EXAMPLE 1 may include the optical flow tube being disposable and the disinfection and detection device being reusable.

In EXAMPLE 3, the fluid catheter system of any of EXAMPLES 1 and 2 may include the disinfection and detection device comprising a clamshell spectrometer.

In EXAMPLE 4, the fluid catheter system of any of EXAMPLES 1-3 may include the optical flow tube comprising at least one sensor for measuring at least one of a flow rate of urine within the system and a temperature of the urine within the system.

In EXAMPLE 5, the fluid catheter system of any of EXAMPLES 1-4 may further include the disinfection and detection device comprising a radio frequency identification (RFID) reader for powering the at least one sensor.

In EXAMPLE 6, the fluid catheter system of any of EXAMPLES 1-5 may further include the disinfection and detection device comprising a radio frequency identification (RFID) reader for reading the at least one sensor.

In EXAMPLE 7, the fluid catheter system of any of EXAMPLES 1-6 may further include the disinfection and detection device being wirelessly connected to cloud services.

In EXAMPLE 8, the fluid catheter system of any of EXAMPLES 1-7 may further include the cloud services comprising at least one of performing spectrometer calibration, storing spectral scan and sensor data, performing urinalysis; performing a sensor fusion algorithm; and providing remote access to collected data.

In EXAMPLE 9, the fluid catheter system of any of EXAMPLES 1-8 may further include the disinfection and detection device comprising a dual spectrometer system and an ultraviolet-C light emitting diode (UVC LED).

In EXAMPLE 10, the fluid catheter system of any of EXAMPLES 1-9 may further include the optical flow tube being optically transparent at a range of wavelengths.

In EXAMPLE 11, the fluid catheter system of any of EXAMPLES 1-10 may further include the range of wavelengths comprising at least one of 255-275 nm, 740-1100 nm, and 1500-2500 nm.

In EXAMPLE 12, the fluid catheter system of any of EXAMPLES 1-11 may further include the optical flow tube comprising a permeable membrane to reject air bubble formation.

In EXAMPLE 13, the fluid catheter system of any of EXAMPLES 1-12 may further include the optical flow tube comprising an acoustic coupler for capturing and propagating acoustic energy to inhibit biofilm formation.

In EXAMPLE 14, the fluid catheter system of any of EXAMPLES 1-13 may further include the optical flow tube comprising an ultraviolet-C (UVC) antireflective coating.

In EXAMPLE 15, the fluid catheter system of any of EXAMPLES 1-14 may further include the optical flow tube comprising a near infrared (NIR) antireflective coating.

In EXAMPLE 16, the fluid catheter system of any of EXAMPLES 1-15 may further include the clamshell spectrometer comprising a reflective interior.

In EXAMPLE 17, the fluid catheter system of any of EXAMPLES 1-16 may further include the clamshell spectrometer blocking ambient light and provides visual spectrum (VIS)-near infrared (NIR) light integration.

In EXAMPLE 18, the fluid catheter system of any of EXAMPLES 1-17 may further include the clamshell spectrometer deterring biofilm growth via ultrasound and providing an ultraviolet-C (UVC) light source to disinfect the fluid catheter system.

EXAMPLE 19 is an optical flow tube for use in a fluid catheter system, the optical flow tube for connecting a catheter tube to a collection tube and including at least one sensor for measuring at least one of a flow rate of urine within the system and a temperature of the urine within the system; a permeable membrane to reject air bubble formation; an acoustic coupler for capturing and propagating acoustic energy to inhibit biofilm formation; and at least one of an ultraviolet-C (UVC) antireflective coating and a near infrared (NIR) antireflective coating; wherein the optical flow tube is optically transparent at certain wavelengths.

EXAMPLE 20 is a fluid catheter system including a disinfection and detection device configured to enclose an optical flow tube for detecting and destroying bacteria within the optical flow tube, the disinfection and detection device further including a dual spectrometer system; and a radio frequency identification (RFID) reader for reading at least one sensor disposed in the optical flow tube, the at least one sensor for measuring at least one of a flow rate of urine within the system and a temperature of the urine within the system; wherein the disinfection and detection device is wirelessly connected to cloud services, the cloud services comprising at least one of performing spectrometer calibration, storing spectral scan and sensor data, performing urinalysis; performing a sensor fusion algorithm; and providing remote access to collected data.

It should be noted that all of the specifications, dimensions, and relationships outlined herein (e.g., the number of elements, operations, steps, etc.) have only been offered for purposes of example and teaching only. Such information may be varied considerably without departing from the spirit of the present disclosure, or the scope of the appended claims. The specifications apply only to one non-limiting example and, accordingly, they should be construed as such. In the foregoing description, exemplary embodiments have been described with reference to particular component arrangements. Various modifications and changes may be made to such embodiments without departing from the scope of the appended claims. The description and drawings are, accordingly, to be regarded in an illustrative rather than in a restrictive sense.

Note that with the numerous examples provided herein, interaction may be described in terms of two, three, four, or more electrical components. However, this has been done for purposes of clarity and example only. It should be appreciated that the system may be consolidated in any suitable manner. Along similar design alternatives, any of the illustrated components, modules, and elements of the FIGURES may be combined in various possible configurations, all of which are clearly within the broad scope of this Specification. In certain cases, it may be easier to describe one or more of the functionalities of a given set of flows by only referencing a limited number of electrical elements. It should be appreciated that the electrical circuits of the FIGURES and its teachings are readily scalable and may accommodate a large number of components, as well as more complicated/sophisticated arrangements and configurations. Accordingly, the examples provided should not limit the scope or inhibit the broad teachings of the electrical circuits as potentially applied to myriad other architectures.

It should also be noted that in this Specification, references to various features (e.g., elements, structures, modules, components, steps, operations, characteristics, etc.) included in “one embodiment”, “exemplary embodiment”, “an embodiment”, “another embodiment”, “some embodiments”, “various embodiments”, “other embodiments”, “alternative embodiment”, and the like are intended to mean that any such features are included in one or more embodiments of the present disclosure, but may or may not necessarily be combined in the same embodiments.

It should also be noted that the functions related to circuit architectures illustrate only some of the possible circuit architecture functions that may be executed by, or within, systems illustrated in the FIGURES. Some of these operations may be deleted or removed where appropriate, or these operations may be modified or changed considerably without departing from the scope of the present disclosure. In addition, the timing of these operations may be altered considerably. The preceding operational flows have been offered for purposes of example and discussion. Substantial flexibility is provided by embodiments described herein in that any suitable arrangements, chronologies, configurations, and timing mechanisms may be provided without departing from the teachings of the present disclosure.

Numerous other changes, substitutions, variations, alterations, and modifications may be ascertained to one skilled in the art and it is intended that the present disclosure encompass all such changes, substitutions, variations, alterations, and modifications as falling within the scope of the appended claims.

Note that all optional features of the device and system described above may also be implemented with respect to the method or process described herein and specifics in the examples may be used anywhere in one or more embodiments.

The “means for” in these instances (above) may include (but is not limited to) using any suitable component discussed herein, along with any suitable software, circuitry, hub, computer code, logic, algorithms, hardware, controller, interface, link, bus, communication pathway, etc.

Note that with the example provided above, as well as numerous other examples provided herein, interaction may be described in terms of two, three, or four network elements. However, this has been done for purposes of clarity and example only. In certain cases, it may be easier to describe one or more of the functionalities of a given set of flows by only referencing a limited number of network elements. It should be appreciated that topologies illustrated in and described with reference to the accompanying FIGURES (and their teachings) are readily scalable and may accommodate a large number of components, as well as more complicated/sophisticated arrangements and configurations. Accordingly, the examples provided should not limit the scope or inhibit the broad teachings of the illustrated topologies as potentially applied to myriad other architectures.

It is also important to note that the steps in the preceding flow diagrams illustrate only some of the possible signaling scenarios and patterns that may be executed by, or within, communication systems shown in the FIGURES. Some of these steps may be deleted or removed where appropriate, or these steps may be modified or changed considerably without departing from the scope of the present disclosure. In addition, a number of these operations have been described as being executed concurrently with, or in parallel to, one or more additional operations. However, the timing of these operations may be altered considerably. The preceding operational flows have been offered for purposes of example and discussion. Substantial flexibility is provided by communication systems shown in the FIGURES in that any suitable arrangements, chronologies, configurations, and timing mechanisms may be provided without departing from the teachings of the present disclosure.

Although the present disclosure has been described in detail with reference to particular arrangements and configurations, these example configurations and arrangements may be changed significantly without departing from the scope of the present disclosure. For example, although the present disclosure has been described with reference to particular communication exchanges, embodiments described herein may be applicable to other architectures.

Numerous other changes, substitutions, variations, alterations, and modifications may be ascertained to one skilled in the art and it is intended that the present disclosure encompass all such changes, substitutions, variations, alterations, and modifications as falling within the scope of the appended claims. In order to assist the United States Patent and Trademark Office (USPTO) and, additionally, any readers of any patent issued on this application in interpreting the claims appended hereto, Applicant wishes to note that the Applicant: (a) does not intend any of the appended claims to invoke paragraph six (6) of 35 U.S.C. section 142 as it exists on the date of the filing hereof unless the words “means for” or “step for” are specifically used in the particular claims; and (b) does not intend, by any statement in the specification, to limit this disclosure in any way that is not otherwise reflected in the appended claims.

What is claimed is:

1. A fluid catheter system comprising:
an optical flow tube for connecting a catheter tube to a collection tube; and
a disinfection and detection device configured to enclose the optical flow tube for detecting and destroying bacteria within the optical flow tube.

2. The fluid catheter system of claim 1, wherein the optical flow tube is disposable and the disinfection and detection device is reusable.

3. The fluid catheter system of claim 1, wherein the disinfection and detection device comprises a clamshell spectrometer.

4. The fluid catheter system of claim 1, wherein the optical flow tube comprises at least one sensor for measuring at least one of a flow rate of urine within the system and a temperature of the urine within the system.

5. The fluid catheter system of claim 4, wherein the disinfection and detection device comprises a radio frequency identification (RFID) reader for powering the at least one sensor.

6. The fluid catheter system of claim 4, wherein the disinfection and detection device comprises a radio frequency identification (RFID) reader for reading the at least one sensor.

7. The fluid catheter system of claim 1, wherein the disinfection and detection device is wirelessly connected to cloud services.

8. The fluid catheter system of claim 7, wherein the cloud services comprise at least one of performing spectrometer calibration, storing spectral scan and sensor data, performing urinalysis; performing a sensor fusion algorithm; and providing remote access to collected data.

9. The fluid catheter system of claim 1, wherein the disinfection and detection device comprises a dual spectrometer system and an ultraviolet-C light emitting diode (UVC LED).

10. The fluid catheter system of claim 1, wherein the optical flow tube is optically transparent at a range of wavelengths.

11. The fluid catheter system of claim 10, wherein the range of wavelengths comprises at least one of 255-275 nm, 740-1100 nm, and 1500-2500 nm.

12. The fluid catheter system of claim 1, wherein the optical flow tube comprises a permeable membrane to reject air bubble formation.

13. The fluid catheter system of claim 1, wherein the optical flow tube comprises an acoustic coupler for capturing and propagating acoustic energy to inhibit biofilm formation.

14. The fluid catheter system of claim 1, wherein the optical flow tube comprises an ultraviolet-C (UVC) antireflective coating.

15. The fluid catheter system of claim 1, wherein the optical flow tube comprises a near infrared (NIR) antireflective coating.

16. The fluid catheter system of claim 3, wherein the clamshell spectrometer comprises a reflective interior.

17. The fluid catheter system of claim 3, wherein the clamshell spectrometer blocks ambient light and provides visual spectrum (VIS)-near infrared (NIR) light integration.

18. The fluid catheter system of claim 3, wherein the clamshell spectrometer deters biofilm growth via ultrasound and provides an ultraviolet-C (UVC) light source to disinfect the fluid catheter system.

19. An optical flow tube for use in a fluid catheter system, the optical flow tube for connecting a catheter tube to a collection tube and comprising:
at least one sensor for measuring at least one of a flow rate of urine within the system and a temperature of the urine within the system;
a permeable membrane to reject air bubble formation;
an acoustic coupler for capturing and propagating acoustic energy to inhibit biofilm formation; and
at least one of an ultraviolet-C (UVC) antireflective coating and a near infrared (NIR) antireflective coating;
wherein the optical flow tube is optically transparent at certain wavelengths.

20. A fluid catheter system comprising:
a disinfection and detection device configured to enclose an optical flow tube for detecting and destroying bacteria within the optical flow tube, the disinfection and detection device further comprising:
a dual spectrometer system; and
a radio frequency identification (RFID) reader for reading at least one sensor disposed in the optical flow tube, the at least one sensor for measuring at least one of a flow rate of urine within the system and a temperature of the urine within the system;
wherein the disinfection and detection device is wirelessly connected to cloud services, the cloud services comprising at least one of performing spectrometer calibration, storing spectral scan and sensor data, performing urinalysis; performing a sensor fusion algorithm; and providing remote access to collected data.
